(19) United States
(12) Patent Application Publication
(10) Pub. No.: US 2010/0305603 A1
(43) Pub. Date: Dec. 2, 2010

(54) Shape-Changing Medical Device, Kit, Method Of Production, And Method Of Use

(76) Inventors: Stevan Nielsen, Rottenburg Am Neckar (DE); Bodo Quint, Rottenburg (DE); Gerd Seibold, Ammerbuch (DE); Ib Erling Joergensen, Haigerloch (DE)

Correspondence Address: INSKEEP INTELLECTUAL PROPERTY GROUP, INC, 2281 W. 190TH STREET, SUITE 200, TORRANCE, CA, 90504, US

(21) Appl. No.: 12/738,623

(22) PCT Filed: Oct. 17, 2007
(86) PCT No.: PCT/EP07/61116

§ 371 Date: Aug. 9, 2010

(51) Int. Cl. A61M 29/00

(52) U.S. Cl. 606/194

(57) ABSTRACT

The present disclosure relates to treatment of disorders in the heart rhythm regulation system and, more particularly, to a medical device for tissue cutting and/or migrating wherein the cutting and/or migrating is at least partly actuated by the swelling of a swellable material. The disclosure furthermore relates to a method of producing such a medical device, a kit of such medical devices, and a method for treating such disorders.

### RELATED APPLICATIONS

This application claims priority to International Patent Application No. PCT/EP2007/061116, International Filing Date 17 Oct. 2007, entitled Shape-Changing Medical Device, Kit, Method Of Production And Method Of Use, which is hereby incorporated herein by reference.

### FIELD OF THE INVENTION

The present invention relates to treatment of disorders in the heart rhythm regulation system and, more particularly, to a medical device for tissue cutting and/or migrating into the tissue, wherein the cutting and/or migrating of the medical device is actuated by a swelling of a swellable material comprised of the device. The invention furthermore relates to a method of producing such a medical device, a kit of such medical devices, and a method for treating such disorders.

### BACKGROUND OF THE INVENTION

The circulation of blood in the body is controlled by the pumping action of the heart. The heart expands and contracts by the force of the heart muscle under impulses from the heart rhythm regulation system. The heart rhythm regulation system transfers an electrical signal for activating the heart muscle cells.

The normal conduction of electrical impulses through the heart starts in the sinoatrial node, travels across the right atrium, the atrioventricular node, the bundles of His and thereafter spread across the ventricular muscle mass. Eventually when the signal reaches the myocytes specialized in only contraction, the muscle cell will contract and create the pumping function of the heart (see FIG. 1).

The electrical impulses are transferred by specially adapted cells. Such a cell will create and discharge a potential over the cell membrane by pumping ions in and out of the cell. Adjacent cells are joined end-to-end by intercalated disks. These disks are cell membranes with a very low electrical impedance. An activation of a potential in a cell will propagate to adjacent cells thanks to the low impedance of the intercalated disks between the cells. While being at the embryonic stage, all heart muscle cells, the myocytes, have the ability to create and transfer electrical signals. During evolution the myocytes specialize and only those cells necessary for maintaining a stable heart-rate are keeping the ability to create and send electrical impulses. For a more thorough explanation of the propagation of electrical signals in the heart, see e.g. Sandöe, E. and Sigurd, B., Arrhythmia, Diagnosis and Management, A Clinical Electrocardiographic Guide, Fachmed AG, 1984.

The heart function will be impaired if there is a disturbance on the normal conduction of the electrical impulses. For instance, atrial fibrillation (AF) is a condition of electrical disorder in the heart rhythm regulation system. In this condition, premature and fast signals irregularly initiating muscle contractions in the atria as well as in the ventricles will be started in ectopic sites, that is areas outside the sinoatrial node. These signals will be transmitted erratically all over the heart. When more than one such ectopic site starts to transmit, the situation becomes totally chaotic, in contrast to the perfect regularity in a healthy heart, where the rhythm is controlled from the sinoatrial node.

Atrial fibrillation is a very common disorder, thus 5% of all patients that undergo heart surgery suffer from AF. 0.4-2% of a population will suffer from AF, whereas 10% of the population over the age of 65 suffers from AF. 160 000 new cases occur every year in the US and the number of cases at present in the US is estimated to be around 3 million persons. Thus, treatment of atrial fibrillation is an important topic.

Typical sites for ectopic premature signals in AF may be anywhere in the atria, in the pulmonary veins (PV), in the coronary sinus (CS), in the superior vena cava (SVC) or in the inferior vena cava (IVC). There are myocardial muscle sleeves present around the orifices and inside the SVC, IVC, CS and the PVs. Especially around the orifice of the left superior pulmonary vein (LSPV) such ectopic sites are frequent, as well as at the orifice of the right superior pulmonary vein (RSPV). In AF multiple small circles of a transmitted electrical signal started in an ectopic site may develop, creating re-entry of the signal in circles and the circle areas will sustain themselves for long time. There may be only one ectopic site sending out signals leading to atrial flutter, or there may be multiple sites of excitation resulting in atrial fibrillation. The conditions may be chronic or continuous since they never stop. In other cases there may be periods of normal regular sinus rhythm between arrhythmias. The condition will then be described as intermittent.

In the chronic or continuous cases, the atrial musculature undergoes an electrical remodelling so that the re-entrant circuits sustain themselves continuously. The patient will feel discomfort by the irregular heart rate, sometimes in form of cannon waves of blood being pushed backwards in the venous system, when the atria contract against a closed arterio-ventricle valve. The irregular action of the atria creates standstill of blood in certain areas of the heart, predominantly in the auricles of the left and right atrium. Here, blood clots may develop. Such blood clots may in the left side of the heart get loose and be taken by the blood stream to the brain, where it creates disastrous damage in form of cerebral stroke. AF is considered to be a major cause of stroke, which is one of the biggest medical problems today.

Today, there are a few methods of treating the problems of disorders to the heart rhythm regulation system. Numerous drugs have been developed to treat AF, but the use of drugs is not effective to a large part of the patients. Thus, there has also been developed a number of surgical therapies.

Surgical therapy was introduced by Drs. Cox, Boineau and others in the late 1980s. The principle for surgical treatment is to cut all the way through the atrial wall by means of knife and scissors and create a total separation of the tissue. Subsequently the tissues are sewn together again to heal by fibrous tissue, which does not have the ability to transmit myocardial electrical signals. A pattern of cutting was created to prohibit the propagation of impulses and thereby isolate the ectopic sites, and thus maintain the heart in sinus rhythm. The rationale for this treatment is understandable from the description above, explaining that there must be a physical contact from myocyte to myocyte for a transfer of information between them. By making a complete division of tissue, a replacement by non-conductive tissue will prohibit further ectopic sites to take over the stimulation. The ectopic sites will thus be isolated and the impulses started in the ectopic sites will therefore not propagate to other parts of the heart.

It is necessary to literally cut the atria and the SVC and the IVC in strips. When the strips are sewn together they will give the impression of a labyrinth guiding the impulse from the sinoatrial node to the atrioventricular node, and the operation was consequently given the name Maze. The cutting pattern is illustrated in FIG. 2 and was originally presented in J L Cox, T E Canavan, R B Schuessler, M E Cain, B D Lindsay, C Stone, P K Smith, P B Corr, and J P Boineau, The surgical treatment of atrial fibrillation. II. Intraoperative electrophysiologic mapping and description of the electrophysiologic basis of atrial flutter and atrial fibrillation, J Thorac Cardiovasc Surg, 1991 101: 406-426. The operation has a long-time success of curing patients from AF in 90% of the patients. However, the Maze operation implicate that many suture lines have to be made and requires that the cuts are completely sealed, which is a demanding task for every surgeon that tries the method. The operation is time consuming, especially the time when the patients own circulation has to be stopped and replaced by extracorporeal circulation by means of a heart-lung machine. Thus mortality has been high and the really good results remained in the hands of a few very trained and gifted surgeons.

The original Maze operation has therefore been simplified by eliminating the number of incisions to a minimum, still resulting in a good result in most cases. The currently most commonly used pattern of incisions is called Maze III (see FIG. 3).

Other methods of isolating the ectopic sites have also been developed recently. In these methods, the actual cutting and sewing of tissue has been replaced by methods for killing myocyte cells. Thus, one may avoid separating the tissue, instead one destroy the tissue by means of heat or cooling in the Maze pattern to create a lesion through the heart wall. The damaged myocyte tissue cannot transfer signals anymore and therefore the same result may be achieved. Still the chest has to be opened, and the heart stopped and opened. Further, the energy source has to be carefully controlled to affect only tissue that is to be destroyed.

A large number of devices have now been developed using various energy sources for destroying the myocyte tissue. Such devices may use high radio frequency energy, as disclosed in e.g. U.S. Pat. No. 5,938,660, or microwaves, ultrasound or laser energy. Recently, devices have been developed for catheter-based delivery of high radio frequency energy through the venous and or arterial systems. However, this has so far had limited success due to difficulties in navigation and application of energy and also late PV stenosis has been reported. Further, devices using cooling of tissue have used expanding argon gas or helium gas to create temperatures of −160° C. Using an instrument with a tip, tissue can be frozen and destroyed.

WO 2006/122961 of the same applicant as the present application discloses a biodegradable tissue cutting device configured for reducing undesired signal transmission in a heart tissue. The cutting action of the biodegradable tissue cutting device is e.g. actuated by means of shape memory materials.

### SUMMARY OF THE INVENTION

Accordingly, some embodiments of the present invention preferably seek to mitigate, alleviate or eliminate one or more deficiencies, disadvantages or issues in the art, such as the above-identified, singly or in any combination by providing a medical device, which changes shape by swelling of a swellable material when said medical device is inserted into a body vessel and/or the heart.

According to embodiments of the invention, there is provided a possibility of cutting through the heart wall in a new manner. Thus, a similar pattern to the Maze III-pattern may also be achieved according to this new manner. However, it may not in all cases be required that all cuts of the Maze III-pattern are made.

An aspect of the invention relates to medical device configured for reducing undesired signal transmission in a body vessel adjacent to the heart and/or a heart tissue by isolating electrical propagation thereof by cutting and/or migrating into said vessel and/or tissue, said medical device is configured for cutting and/or migrating by changing its shape. The medical device comprises a swellable material which is capable of actuating said change of shape via swelling, in embodiments due to water absorption, by said swellable material.

Another aspect of the invention relates to a kit of shape-changing medical devices as described herein for treatment of disorders in the heart rhythm regulation system, said kit comprising said shape-changing medical devices, which each has a first delivery and a second delivered shape, wherein the device in the first shape has such dimensions as to be insertable to a desired position within the vascular system, and wherein the device is capable of changing shape to substantially the second shape when located at said desired position, which strives to a diameter that is larger than the diameter of the vessel at the desired position, whereby the device will become embedded into the tissue surrounding the vessel at the desired position and create fibrosis and/or create scar tissue in said tissue in order to prevent it from transmitting electrical signals, wherein at least one of the shape-changing devices is adapted to be inserted to a desired position at the orifice of a pulmonary vein in the heart, the pulmonary vein adjacent said orifice, or inside the right or left atrium and at least one of the shape-changing medical devices is adapted to be inserted to a desired position in the coronary sinus, and wherein said medical devices comprise a swellable material which is capable of actuating said change of shape via swelling due to water absorption of said swellable material.

Yet an aspect of the present invention relates to a method for treatment of disorders in the heart rhythm regulation system using a medical device comprising a swellable material, the method comprising inserting the medical device in a temporary delivery shape through the vascular system into a body vessel adjacent to the heart and/or into the heart; allowing said swellable material to swell by absorbing water, said swelling at least contributing to changing the shape of the medical device, from said temporary delivery shape via an expanded delivered shape to a further expanded shape, extending at least beyond an outer surface of said tissue and/or vessel, thereby creating cutting action configured for cutting said heart tissue and/or said body vessel, or creating migrating action configured for migrating said medical device into said heart tissue and/or said body vessel, thereby reducing undesired signal transmission in said heart tissue and/or said body vessel by cutting said heart tissue and/or said body vessel by means of the medical device configured therefore.

Another aspect of the invention relates to a method of preparing a medical device, the method comprising the steps of:
providing a swellable material
transforming said swellable material into a medical device configured for reducing undesired signal transmission in a body vessel adjacent to the heart and/or a heart tissue by isolating electrical propagation thereof by cutting and/or migrating into said vessel and/or tissue.

In another aspect, a method of delivering a medical device in a body tissue is provided. The method comprises providing an endotheliasation agent for said medical device, delivering the medical device to a target location, contacting said body tissue at said target location with said medical device and said endotheliasation agent, and anchoring said medical device at said target location by a layer of endothelia promoted by said endotheliasation agent.

In an aspect of the invention, the use of an endotheliasation agent for anchoring a medical device in body tissue is provided.

In the context of the present invention, the term “swelling” relates to volumetric expansion of a material due absorption of water. Such absorbed water may e.g. be water absorbed from the blood of a mammal.

In the context of the present invention, the term “swellable material” relates to a material which is capable of expanding its volume in the range of 0.1-1000% relative to its dry state when exposed to the swelling test described herein, and preferably the swellable material is able to expand its volume in the range of 1-500%, such as 5-250%, and even more preferably in the range of 5-100%, such as from 10-70%. Some designs require less expansion of the swellable material, e.g. in range of 0.1-40% relative to its dry state when exposed to the swelling test described herein, and preferably in the range of 1-25% such as 2-10%.

In the context of the present invention, the “swelling test” consists of measuring a parameter, e.g. volume, length or diameter, before swelling of the medical device or the material to be tested. Then the medical device or the material is incubated in an isotonic phosphate buffer for one week at a fixed temperature, which is 20° C. if nothing else is mentioned. Immediately after the one week incubation, the parameter is measured again and the change is calculated.

In the context of the present invention, the term “isotropic swelling” relates to the swelling of a material which exhibits a uniform, three-dimensional expansion by swelling. For a uniformly expanding material the value of the expansion in one direction will be correlation with the third root of the volume expansion. For example, at a 100% volume expansion the length of a wire will expand 25.99%.

In the context of the present invention, the term “anisotropic swelling” relates to the swelling of a material which exhibits non-uniform expansion, i.e. the material has a higher relative expansion in one dimension than in another dimension. Anisotropic expansion may e.g. be obtained using certain production processes or using certain fillers.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic view of the transmission of electrical signals in the heart;

FIG. 2 is a schematic view of a pattern of cutting tissue of the heart wall according to the Maze-procedure for treating disorders to the heart rhythm regulation system;

FIG. 3 is a schematic view of a simplified pattern according to the Maze III-procedure, wherein the heart is seen from behind;

FIG. 4, comprising FIGS. 4a and 4b, is a schematic illustration of a medical device comprising a ring-like structure;

FIG. 5, comprising FIGS. 5a and 5b, is a schematic illustration of a medical device comprising a polygon-like structure;

FIG. 6, comprising FIGS. 6a and 6b, is a schematic illustration of a medical device comprising a spiral-like structure;

FIG. 7, comprising FIGS. 7a, 7b and 7c, is a schematic illustration of a medical device comprising a slotted tube;

FIG. 8, comprising FIGS. 8a and 8b, is a schematic illustration of a medical device comprising a braid-like structure;

FIG. 9 is a schematic illustration of a medical device comprising a bi-ring including a first swellable material and a second material;

FIG. 10, comprising FIGS. 10a and 10b, is a schematic illustration of a medical device comprising a filled tube;

FIG. 11, comprising FIGS. 11a, 11b and 11c, is a schematic illustration of a medical device comprising a ring of biodegradable swellable material and a non-biodegradable part; and

FIG. 12 is a schematic illustration of a medical device comprising a main body comprising a non-swellable material and elements of swellable material.

### DETAILED DESCRIPTION OF THE INVENTION

Some embodiments of the present invention relate to a medical device configured for reducing undesired signal transmission in a body vessel adjacent to the heart and/or a heart tissue by isolating electrical propagation thereof by cutting and/or migrating into said vessel and/or tissue, said medical device is configured for cutting and/or migrating by changing its shape, wherein said medical device comprises a swellable material which is capable of actuating said change of shape via swelling due to water absorption of said swellable material.

In the context of the present invention, the terms “cutting and/or migration” or “cut and/or migrate” relates to the expanding movement of the medical device in which it penetrates through the wall of the body vessel and/or heart chamber where is has been inserted. The expanding movement preferably positions at least a portion of the medical device in the tissue adjacent to the body vessel and/or heart chamber where was originally inserted.

A lesion created by the medical device will be healed with fibrous tissue, which is unable to transmit electrical signals. Thus, it is preferred that the medical device is capable of penetrating sufficiently deep into the tissue surrounding the body vessel or chamber, where it was inserted, to create fibrous tissue which interrupt the coupling between cells that transmit erratic electrical signals.

In some embodiments of the invention, said medical device is a tissue cutting device which is capable of penetrating the vessel tissue and/or heart tissue adjacent said body vessel.

In some embodiments the medical device may be is structured and arranged to be inserted in a temporary delivery shape through the vascular system into a body vessel adjacent to the heart and/or into the heart and subsequently to be subjected to a change of shape, from said temporary delivery shape via an expanded delivered shape to a further expanded shape, extending at least beyond an outer surface of said vessel and/or tissue, in order to create cutting action configured for cutting said heart tissue and/or said body vessel.

In some embodiments of the invention, the medical device may, when it has been inserted in its temporary delivery shape, the be manipulated to change from its temporary delivery shape to a final delivery shape, which will be subjected to a change of shape, from said final delivery shape via an expanded delivered shape to a further expanded shape, extending at least beyond an outer surface of said vessel and/or tissue, in order to create cutting action configured for cutting said heart tissue and/or said body vessel. In other embodiments the temporary delivery shape is the final delivery shape.

The cutting and/or migration may be performed continuously after placement of the medical device, until a final shape or position of the device is accomplished.

The change of shape which the medical device undergoes may be any change of shape. A preferred change of shape is expansion and even more preferred is a radial expansion, e.g. towards the wall of the vessel or chamber where the medical device has been inserted.

In an embodiment of the invention, the medical device, and particularly the swellable material, is so arranged that at least one dimension of the medical device has expanded in the range of approximately 5-500% relative to its pre-swelling state after having been incubated in isotonic phosphate buffer at 20° C. for one week. It is preferred that the medical device has expanded in the range of approximately or exactly 10-200%, such as approximately or exactly 10-150%, and it is even more preferred that the medical device has expanded in the range of approximately or exactly 15-100%, such as approximately or exactly 20-80% relative to its pre-swelling state.

In some embodiments of the invention, the pre-swelling state of the medical device or the swellable materials is its dry state.

It may also be preferred that the medical device, and particularly the swellable material, is so arranged that at least two dimensions of the medical device expand in the range of approximately or exactly 5-500% relative to its pre-swelling state after having been incubated in isotonic phosphate buffer at 20° C. for one week. Again it is preferred that the medical device has expanded in the range of approximately or exactly 10-200% in each of the at least two dimensions, such as approximately or exactly 10-150%, and it is even more preferred that the medical device has expanded in the range of approximately or exactly 15-100%, such as approximately or exactly 20-80% relative to its pre-swelling state.

The expansion in at least one dimension or at least two dimensions preferably comprises radial expansion, i.e. expansion of the medical device towards and into the body vessel wall and/or the heart chamber wall.

The medical device, and particularly the swellable material, is typically configured and arranged so that the medical device reaches its maximum expansion in the range of 1 hour-3 months, preferably in the range of 1 day-1 month, and even more preferred in the range of 2 day-10 days.

In some embodiments of the present invention, it is preferred that the outer diameter of the medical device, when it is maximally expanded, is in the range of 10-50 mm, preferably in the range of 20-40 mm, and even more preferred in the range of 25-35 mm.

Several types of swellable materials may be employed in embodiments of the present invention. It is preferred that the swellable material is biocompatible and preferably also biodegradable.

The swellable material may comprise a swellable polymer and preferably a hydrophilic, swellable polymer. Such a swellable polymer may comprise a polar non-charged polymer and/or it may comprise a charged polymer such as a polyelectrolyte.

Examples of useful polyelectrolytes are poly(acrylic acid) (PAA) and poly(aspartic acid). Poly(aspartic acid) is the biodegradable and thus especially preferred.

Examples of useful non-charged swellable polymers are e.g. poly(ethylene glycol) (PEG), poly(vinyl-pyrrolidone) (PVP), N-(2-hydroxypropyl)methacrylamide (HPMA), polysuccinimide, poly(vinyl-alcohol), starch, and cellulose derivatives such as ethers or esters.

PEG has the advantage that it is biodegradable. PVP offers almost no metabolic interference when exposed to the body. HPMA oligomers up to MW around 30 kDa are not recognized as foreign molecules and capable to pass the body with no metabolic interference. Starches and certain cellulose derivatives (e.g. hydroxypropyl cellulose) are also biodegradable.

The swellable polymer may in some embodiments e.g. comprise a copolymer, it may comprise a blend of different polymers, or it may comprise cross-linked polymers.

The materials of the device according to some embodiments may support the cutting and/or migrating effect advantageously, e.g. by enhancing a cutting effect of the device, such as providing a faster continuous cutting/migrating and scar tissue creating than devices of other, more inert materials not enhancing any cutting and/or migrating effect by the nature of its material.

In some embodiments, the swelling property of a swellable polymer may be related to the swellable polymer itself or swelling-promoting additives incorporated in a polymer matrix of the swellable polymer.

The physical swelling due to water absorption is often attributed the embedding of water molecules between polymer chains of the swellable polymer. The polymer chains need to be polar and may have salt-like dissociation elements or capabilities to interfere with hydrogen bridge bonds. Due to this effect primary polar fixation forces between polymer chains are reduced or isolated. Macroscopic this effect may cause a loss of stiffness and increase of volume.

In some embodiments, swellable polymers may comprise cross-links between the polymer chains in order to provide maximum hydrophilicity but to avoid or delay the dissolubility of the swellable polymer.

In some embodiments, charged hydrophilic polymers segments are able to change the osmotic environment inside the polymer and therefore yield an additional expansion effect by osmotic pressure. Therefore polyelectrolyte like polymer segments of some embodiments may use osmotic effects to increase swelling volume or if the volume is restricted by the polymer structure pressure to enhance mechanical and dimensional stability.

In some embodiments of the invention the swellable material and/or the swellable polymer additionally comprises a swelling-promoting additive. The swelling-promoting additive may e.g. comprise an inorganic additive and/or an organic additive.

The swelling-promoting additive may be an acid, thus creating a locally acidic environment when reacting with water. The swelling-promoting additive may be basic, thus creating a locally basic environment when reacting with water. It is furthermore envisioned that the swelling-promoting additive may be pH neutral, thus not affecting the pH of the local environment when reacting with water.

It is preferred that the swelling-promoting additive is biocompatible and preferably also biodegradable.

In some embodiments of the invention, the swellable material may comprises a polymer matrix mixed with one or more swelling-promoting additives. The polymer matrix may e.g. be a non-swellable polymer.

The swelling-promoting additives may advantageously be used for controlling the rate absorption of water and thus the rate of swelling for the swellable material. In embodiments where the medical device does not contain a swellable polymer, swelling-promoting additives are also responsible for the swelling action. Another advantage of the swelling-promoting additives is that they may change the local pH of the swellable material and optionally also the medical device and may thereby accelerate the degradation, and particularly the hydrolysis, of the swellable material and the medical device. In this manner a cutting and/or migrating effect may be enhanced by the degradation.

Examples of useful inorganic additives are inorganic oxides, salts, or surface structures such as clays. The inorganic substances may be dispersed in the polymer matrix. The polymer matrix is preferably made from polymers which have elastic and tough mechanical properties.

The polymer matrix may e.g. be a biodegradable polymer. A number of useful biodegradable polymers are mentioned in WO 2006/122961, which is incorporated herein by reference in its entirety for all purposes. Examples of biodegradable polymer matrices are e.g. randomly polymerized copolymers of D,L-lactide, lactide-caprolactone or TMC (tri methyl carbonate).

The swelling effect of the swellable material comprising a polymer matrix comprising a swelling-promoting agent may be caused by absorption of water by the swelling-promoting agent and/or by chemical reactions between the water and the swelling-promoting agent.

In a preferred embodiment of the invention, the inorganic additive comprises a salt or an oxide of calcium. Examples of useful calcium compounds are shown in Table 1.

TABLE 1
The swelling potential of inorganic calcium compounds.

| | | Molar | | | |
|---|---|---|---|---|---|
| Inorganic | Reaction | volume | | | |
| additive | product | [g/mol] | Reaction | A/R | Comments |
| CaO | | 16.79 | ↓ | | Initial swelling |
| | | | | | material |
| | −> | 33.68 | | 101% | strong alkaline |
| | Ca(OH)2 | | | | reaction |
| | −> | | | | Soluble |
| | (CaCO3) | | | | |
| CaHPO4 | | 46.59 | ↓ | | Initial swelling |
| | | | | | material |
| | −> | 74.50 | | 60% | neutral pH, |
| | CaHPO4 × | | | | almost |
| | H2O | | | | no solubility |
| CaSO4 | | 45.99 | ↓ | | Initial swelling |
| | | | | | material |
| | −> | 74.21 | | 61% | mild acidic, |
| | CaSO4 × | | | | low solubility |
| | 2 H2O | | | | |

A/R is the relative volume expansion between the starting material and the reaction product, based on their molar volumes.

Silicates are another group of useful inorganic additives which offer swelling or expanding properties in combination with minor toxic properties. Silicates e.g. Clays or fumed silica offer physical swelling by aligning water molecules between intensive charged surface structures. Natural or synthetic layered silicate structures offer a significant change in volume due to their high surface area and their electrostatic charge in the presence of electrolytes.

Clays are also useful inorganic additives for reinforcement. In order to maximize swelling properties of the resulting swellable material, the clay may be embedded in the polymer matrix in poorly dispersed, non-exfoliated state and higher contents above 7% into the polymer matrix. An example of useful clay is Bentonite (see the European Pharmacopoeia 4) where 6 g dry Bentonite expands to 98 ml via suspension in water.

A swellable material comprising a swelling-promoting additive and a polymer matrix may be prepared by compounding, which is well known process in polymer industry. The compounding process is typically based on a twin screw compounder which is fed with weight dosages to realize granulate and powder (filler) supply. A suitable additive to increase processability and final mechanical properties of the compound is for example stearic acid. The content range of this additive is typically 0.5-7% by weight of the filler. Higher contents in the case of stearic acid may be used to modify the polymer with additional hydrophobic properties. In the ideal process, the filler is already pre coated onto the fillers surface.

In addition or alternatively, other tenside-like additives, which are stable at the processing temperature of the matrix polymer, may also be used instead of stearic acid. The compounding process needs to be designed for minimal moisture contact. Technical solutions to achieve this goal starts with providing carefully pre-dried ingredients, the use of dry inert gases to isolate melt and mixing areas from the environment and may also avoid the typical cooling of the compound in a water bath prior to granulation.

The swelling forces created by the inorganic additive may deform and expand even hard and rigid polymer matrices like PLLA. In order to reduce brittle behaviour of the polymer matrix, additional additives like clays may be compounded into the polymer matrix. The content range of this clay additive is between 4-10% relative to the matrix polymer content. The inorganic additive may be incorporated into the polymer matrix in the same compounding step as the clay additive.

In an embodiment of the present invention, the swellable material comprises inorganic additive in an amount of 5-40% by weight and polymer matrix in an amount of 60-95% by weight, and preferably inorganic additive in an amount of 10-30% by weight and polymer matrix in an amount of 70-90% by weight. Such combinations offer both a useful swelling effect and a sufficient integrity of the resulting swellable material.

In contrast to shape memory polymer designs, designing the polymer construction with swelling inorganic additives enables to heat treat and shape the polymer design at temperatures above the glass-transition temperature (T_{g}) of the polymer matrix in order to realize maximum crystallinity of the polymer matrix. The T_{g} of the used polymer matrices is preferably significant above 37° C. in order to realize more ideal elastic mechanical behaviour and high e-modulus.

Increased crystallinity of the polymer decreases related viscoelastic effects. Similar sized medical device designs can therefore provide more effective radial force or may be carried out by using more filigree structures, thanks to this advantageous effect.

The swellable material may also comprise a hydrogel. Hydrogels are networks of polymer chains that are water-insoluble, sometimes found as a colloidal gel in which water is the dispersion medium. Hydrogels are superabsorbent (they even may contain over 99% water) natural or synthetic polymers. Hydrogels typically possess also a degree of flexibility very similar to natural tissue, due to their significant water content. Depending on a degree of water-insoluble segments of the polymer, which may be realized by chemical cross links or water insoluble polymer segments this hydrogels offer lower volume increase (or water uptake) and improved mechanical rigidity.

The hydrogel typically comprises one or more hydrogel forming components. Typically, at least one of said one or more hydrogel forming components is selected from the group consisting of a swellable polymer as described above and/or of natural polymers like a chinoline (lecithin), a liposome, a protein, a nucleic acid, and any combination thereof.

Some useful hydrogel forming components are preferably able to form a gel-like consistency when mixed with water and typically have a high hydrophilicity and/or water solubility.

In preferred embodiments of the invention, the hydrogel forming components are cross-linked to improve the coherence and optionally also the elasticity of the resulting hydrogel. The cross-linking may involve cross-links by covalent bonds and/or cross-links by polar interaction, i.e. ionic interaction or hydrogen bonding.

The cross linking may e.g. be achieved by oxidation (e.g. using peroxides), high energy radiation, or chemical interconnection (e.g. by chemical links created with bi or multifunctional mono- or oligomers in a chemical process step). An example for crosslinked hydrogels is the Pro/Peg™ family from Neomend.

In an embodiment of the present invention, the medical device is biodegradable, and thus capable of dissolving and/or degrading once it has been implanted in the body. Useful biodegradable materials can be found in WO 2006/122961.

In an embodiment of the invention, the swellable material contains a poly(lactide)/poly(ethyleneglycole) copolymer hydrogel, which e.g. is commercially available under the name Polyshield® AK03-AK08 from Akina Inc., West Lafayette, Ind., USA. Other useful biodegradable hydrogels are e.g. Pro/PEG™ by Neomend which is based on cross-linked PEG and Polyactive™ OctoPlus NV which is based on a PEG/PBT copolymer system. Both examples are hydrogel families allowing customization.

One or more useful monomers from which hydrogels can be prepared may be selected from the group consisting of acrylates, such as (BHCM)butylhydroxycyclomethylacrylate, (BMA) butyl methylacrylate, (DAA) Diacetoneacrylamide, (DMAA) dimethylyacrylamide, (EOEMA) ethoxyethylmethylacrylate, (GMA) gylcerylmethylacrylate, (HBMA) hydroxybutylmethylacrylate, (HEMA) hydroxyethylmetacrylate, (HEA) hydroxyethylacrylate, (MA) methacrylic acid, (AA) acrylic acid, (MMA) methylmethacrylate; (MPE) methylpropanic acid ester; (PC) phosphorylcholine; (VA) vinylalcohol; (VBL) vinylbutyrolactam; (VP) N-Vinylpyrrolidone; and any combination thereof.

Such monomers can e.g. be polymerized by the radicalic or ionic polymerization mechanisms. This is typically used for synthesis of the material used for contact lenses.

In preferred embodiments of the invention, the swellable material comprises one or more acrylic based hydrogels, which are well known for their use in soft contact lenses. Since there is a huge amount of acrylic monomers this raw materials can be used to offer and to fine tune hydrogel properties. Another advantage of acrylic based hydrogel systems is that they can be realized in a fast and of cause also one single chemical synthesis step.

Acrylic based hydrogels can directly be synthesized by free radical polymerization, for example 2-hydroxyethyl methacrylate (HEMA) and α-tocopheryl methacrylate (VEMA). The hydrogel containing 20 wt % of VEMA and 80% HEMA is showing an equilibrium water content in the range of hydrogel networks at any pH. The swelling follows the Fick's law, indicating that water absorption is controlled by diffusion only. The values of diffusion coefficients for the VEMA-containing hydrogel are lower than those of poly-HEMA in any medium. Surface characterization of the VEMA-containing hydrogel showed a decrease of surface energy of the solid owing to a decrease of the polar component mainly. The application of fine powdered Xerogel (Journal of Materials Science: Materials in Medicine, Volume 10, Number 10-11, 11 Oct. 1999, pp. 641-648(8): Resorbable polyacrylic hydrogels derived from vitamin E and their application in the healing of tendons), a commercial product based on this polymer type physically loaded with Vitamin E, showed very fast and positive response to activate tissue regeneration capacity.

Some of the mentioned monomers may be used to build oligomer segments which are further used in degradable hydrogel systems since they offer the possibility to be separated inside the body through the kidneys (VP,VA). Other materials which offer double bonds or other functional groups like chinolines provide polymerization possibilities towards macromolecular systems and the ability to be metabolized.

In some embodiments the invention it is therefore preferred that either the medical device, or only the swellable material, essentially consists of materials having degradation products which have a molar weight of at most 30 kDa, preferably of at most 25 kDa, and even more preferably of at most 20 kDa. It is particularly preferred that substantially all of the degradation products can be filtered out of the blood by the kidneys and excreted with the urine. This is advantageous with regard to pollution load or effect on the patient.

It is furthermore envisioned that photo initiated polymerization may be used inside the body and even in the presence of blood flow. In U.S. Pat. No. 6,475,508, which is incorporated herein by reference in its entirety, a method is described that uses PEG (PVOH) polymers, to provide water/blood solubility—flanking with PLA oligomers to provide water lability and tetraacrylate termini. Eosin may be used as photoinitiator to be excited with UV light.

Another way of creating hydrogel forming polymers is the chemical modification of polymers to polymer derivates, which is a well know process in the polymer industry. These modification techniques may be used to turn polymer chains towards more hydrophilic or even water soluble characteristics. An example is the modification of purified cellulose to yield hydroxypropyl cellulose, which may be used as a hydrogel in medical products, such as the medical products described herein.

In some embodiments of the invention, the swellable material comprises a mixture of collagen and a further material. The further material may be any of the other swellable materials mentioned herein, e.g. PVA.

Blends of collagen with PVA will create a stable hydrogel. Such blends can for instance be realized by decreasing the pH of an aqueous reaction mixture comprising collagen and PVA. This is an example for blending water soluable polymers to yield a hydrogel. In this case, PVA binds to soluble collagen around pH 3. No reaction occurs in presence of salts because they interfere with electrostatic charged functional groups. This polymer blend may be optimized with respect to its biological stability by chemical cross linking with glutaraldehyde, which forms covalent bonds with primary amino groups of collagen.

Another useful collagen blend is PAA/collagen which was observed as modification layer applied to PAA coated surfaces. The surfaces are exposed to collagen resulting in a cell-supporting collagen layer being attached to non-biocompatible materials such as PET. This surface treatment allows growth of endothelial cells on PET vascular grafts without major implication.

HEMA/collagen blends was developed to combine mechanical properties and degradability of poly(HEMA) with biocompatibility of collagen. In the form of a hydrogel, the blend has been utilized as a drug carrier system that target damaged tissue while releasing controlled amounts of the drug. HEMA/collagen may be used as a material in some embodiments constituting at least partly a medical device.

Another group of type of hydrogel comprises PVP (poly(vinylpyrrolidone)). PVP-comprising hydrogels are reported as favourable substitute to silicon based implants and have been used in breast implants. Hydrogel filled implants are water based and does not show negative body reactions known from silicone oils. The hydrogel comprising PVP preferably has the capability to pass the body without significant metabolic interference through the kidneys.

Hydrogels comprising hydrophilic cellulose derivatives, such as hydroxypropyl cellulose, are also useful and are capable of degrading in the body without significant negative response.

Some of the specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art.

The terminology used in the detailed description of the embodiments illustrated in the accompanying drawings is not intended to be limiting of the invention. In the drawings, like numbers refer to like elements.

In some embodiments of the invention, a medical device has an elongated tubular shape, e.g. if the medical device contains braid-like structure or spiral-like structures. Normally the axial length of such elongated tubular medical device is at least as long as the diameter of the medical device, preferably at least 50% longer than the diameter, and even more preferably at least 100% longer than the diameter of the medical device. Normally, the length of such medical device is shorter than 1000% of its diameter.

In some embodiments of the invention, the medical device comprises a ring-like structure. An exemplary embodiment of this is illustrated in FIGS. 4a and 4b. Here, ring-structured medical devices (40) comprising a swellable material (41) are shown. The swelling of the swellable material both increases the ring thickness as well as the outer diameter of the ring. The ring-structured medical device (40) may be inserted in a delivery catheter in a longitudinal, temporary delivery shape (not shown) and locked into its final delivery shape at the target area, e.g. by suitable restriction elements, such as a sheath. Upon delivery, the ring is released from the delivery catheter for implantation and expands to a first expanded shape, e.g. due to a shape memory effect and/or resiliently. After implantation the ring will start expanding due to swelling and will ultimately cut into and/or migrated through the walls of the vessel or chamber where it has been inserted.

One way of implanting the ring-structured medical device having it in a folded, temporary delivery shape during the insertion and unfolding it when it reaches the target area to obtain its final delivery shape. Another possibility is to insert the medical device in a straight configuration and lock it in the vessel using a form fitting (42 of FIG. 4a) to tie its ends together and thus obtaining its final delivery shape. An alternative to the fitting is having a wire (43) in one end of the ring-structured medical device (40) and inserting the wire (43) through a hole (44) perforating the other end of the ring-structured medical device (40), thus tying the ends of the medical device together.

A medical device having a braid-liked structure typically comprises at least 2 wires which have been interwoven around a core. Commercially available braiding apparatuses e.g. Fine wire braider K 80/16-1K, Körting Nachf. Wilhelm STEEGER GmbH & Co. Germany can be used for the braiding process. Once braided, the wires may be shape-set by performing a heat setting procedure in a standard convector oven or in a vacuum oven at a specified temperature, such as at temperatures in the range of approximately 40 to 120° C. as for certain shape memory polymer wires nut not limited by Tg as for those wires, while fixed on the core. After the heat treatment the core will be removed and the medical device will be cut into a suitable length.

The wire used for the braiding process may comprise swellable material and may even completely consist of swellable material.

The cross-section of the wire may e.g. be circular, oval, rectangular, or any other shape suitable for braid-like structures. In order to obtain an advantageous cutting effect, the cross-section of some embodiments of the invention may comprise at least at a portion of the device a substantially sharp-edged cutting form oriented in the desired cutting/migration direction of the medical device, e.g. radially outwards for a ring shaped medical device. In some embodiments of the present invention, the wire contains several layers, and at least one layer thereof comprises or essentially consists of swellable material, at least along partly sections thereof. Another layer of the wire may essentially consist of non-swellable material.

In some embodiments, the medical device may swell three dimensionally, e.g. both radially and longitudinally, i.e. at the same time in several directions. Due to the construction or the shape of the medical device, the degree of swelling may be different in different directions of swelling.

The wires used in context of the present invention typically have cross-sectional dimensions, such as a diameter, in the range of 0.1-1 mm, preferably in the range of 0.2-0.75 mm, and even more preferably in the range of 0.3-0.6 mm.

Useful target areas for the medical device are e.g. a body vessel adjacent to the heart or a heart atrium. The target area may e.g. be an orifice of a pulmonary vein in the heart, the pulmonary vein adjacent said orifice, the right or left atrium, the coronary sinus or the superior or inferior vena cava.

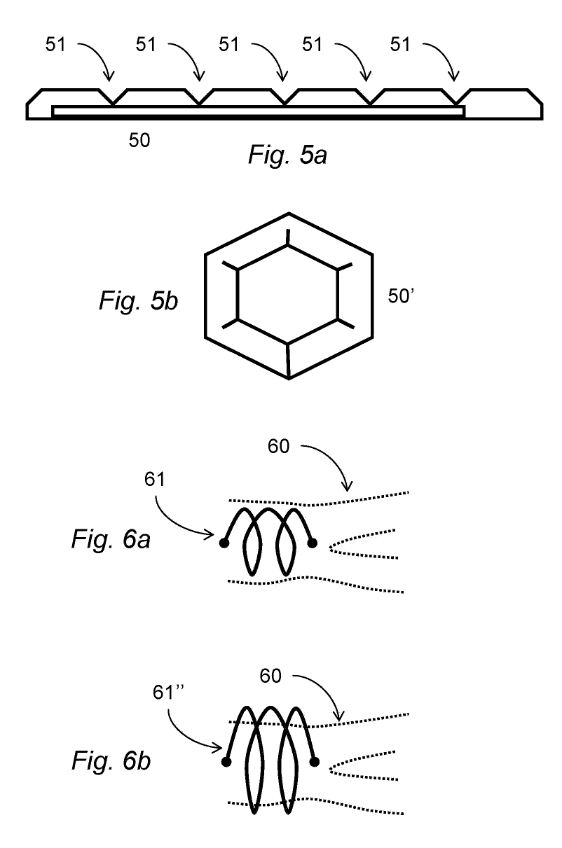

In some other embodiments of the invention, the medical device comprises a polygon-like structure. An example of this is schematically illustrated in FIGS. 5a and 5b. This medical device may e.g. be inserted in a longitudinal, temporary delivery shape (50) and manipulated into its final delivered shape (50′) at the target area. The medical device comprises indents (51) allowing it to be folded into a polygon. The medical device may e.g. contain a form fitting (see 42 of FIG. 4a) for locking its final delivery shape. After the insertion and the manipulation, the polygon-structured medical device will be formed and fixed in the target area. The medical device is preferably adjusted to fit exactly the geometry of the target area (e.g. the atria). Once the medical device has been inserted in its final delivery shape, it is preferably overgrown by endothelial cells, and will start swelling by absorption of water.

It is envisioned that the medical device according to some embodiments of the invention may comprise other structures such as a circular structure, an ellipsoid, or a bone-structure.

In yet another embodiment of the invention, the medical device comprises a spiral-like structure.

This is schematically illustrated in FIGS. 6a and 6b. In FIG. 6a a medical device (61) comprising a swellable material is inserted into its target area (60) (e.g. an atrium) in its temporary delivery shape. The dotted lines represent the walls of the vessel or chamber of the target area (60). The medical device (61) may change shape from its temporary delivery for to an expanded delivery form (not shown), e.g. by elastic self-expansion. The medical device will also expand due to the water absorption and swelling of the swellable material, thus obtaining its further expanded shape (61″), where it cuts and/or migrates into and/or through the wall of the target area.

A medical device according to some embodiments of the invention may be positioned in its temporary delivery shape or its final delivery shape at the target area, such as an atrium, or any other part of the heart or blood vessel, in which a tissue cutting action is desired. The medical device may then be forced to expand into an expanded delivery shape. This expanded delivery shape may then be the starting point for the further change of shape, which change of shape provides the cutting and/or migration action in said tissue to be treated. The change of shape from the temporary delivery shape into the expanded delivery shape may for example be actuated by a spring function incorporated in the medical device, an inflatable and/or expandable balloon, and/or a shape memory effect. When the medical device is positioned in a desired position and in its expanded delivery shape, the swelling may start, and the cutting device is transformed from the expanded delivered shape to the further extended shape, at least partly thanks to the swelling.

The medical device comprising a spiral-like structure is typically prepared from a wire. The wire is wound around a core, fixed and afterwards heat-set using a standard convector oven or in a vacuum oven at a specified temperature. After the heat treatment the core will be removed and the medical device will be cut into a suitable length—typically in the range of 10-60 mm. The used wire may be one of the wires described above.

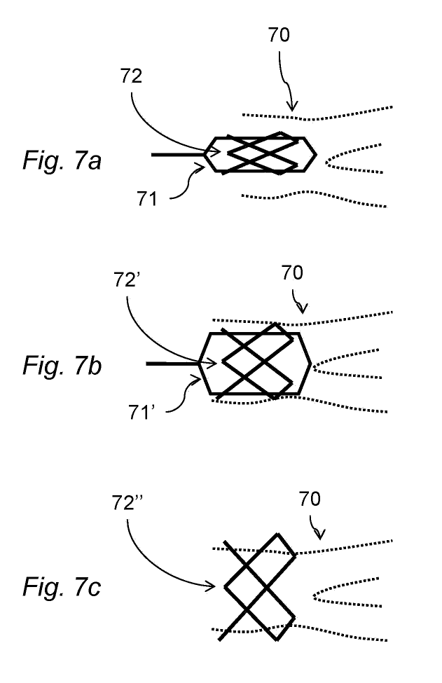

In a further some embodiments of the invention, the medical device comprises a slotted tube. This is illustrated in FIGS. 7a, 7b and 7c. A slotted tube containing swellable material (72) has been inserted at the target area (70) in its temporary delivery shape and an expandable balloon (71) has been positioned inside the slotted tube (72). The balloon is then expanded (71′) and actuates the expansion of the slotted tube into its expanded delivery shape (72′), after which the balloon may be removed. The final change of shape into the further expanded shape (72″) is at least partly actuated by swelling of the swellable material of the slotted tube.

The slotted tube medical device (72) may e.g. be made of a tube of swellable material, wherein a pattern is cut using a laser, a water jet or similar cutting tools. The pattern is designed to be expandable similar to a balloon expandable stainless steel stent. The pattern may have various forms suitable for a desired expansion ratio. The implantation of the slotted tube medical device will be approximately the same procedure as for a balloon expandable stent. The slotted tube is preferably overgrown by endothelial cells, before it expands by swelling in the target area.

The implantation may e.g. be performed using an external force, such as a balloon catheter. After fixation and overgrowth with endothelia cells, the medical device starts to swell and expand. An assemblage of several balloons may be useful if a medical device comprising an oval structure has to be implanted.

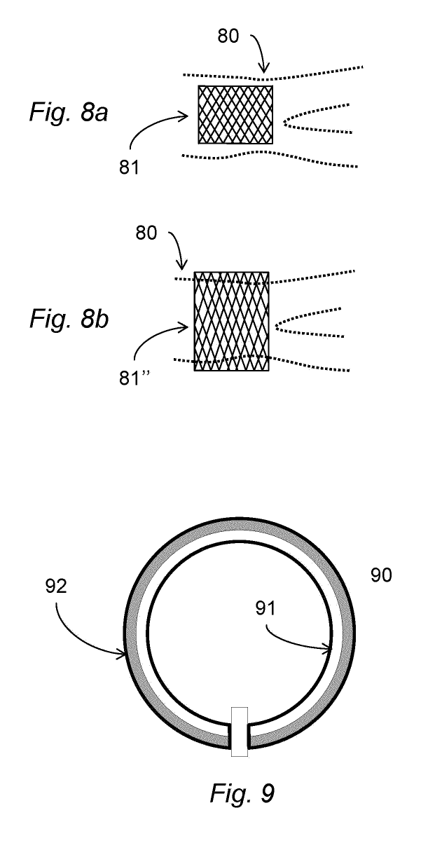

In some embodiments of the invention, the medical device comprises a braid-like structure. See FIG. 8 for a schematic illustration, where a tubular braided structure is shown. The braid-like structured medical device (81) comprises swellable material and is inserted at its target area (80) in its temporary delivery shape. The medical device may for instance expand to its expanded delivery shape (not shown) due to its own elastic properties, a shape memory effect, and/or due to unfolding by a mechanical force e.g. by a balloon to the delivered shape. When the medical device starts to change shape from the temporary delivery shape into the expanded delivery shape, the swellable material may start to absorb water and swell. The swelling results in changing the shape of the medical device from the expanded delivery shape to the further expanded shape and results in the penetration through the wall of the target area (the dotted line of FIGS. 8a and 8b) and into the adjacent tissue.

Overgrowth with endothelial cells may ensure that the medical device will expand into its further expanded shape in a desired way, and that the cutting/migrating action will be performed in the way intended when the medical device was positioned.

It is furthermore envisioned that medical device may comprise at least two structures selected from the group consisting of a ring structure, a polygon, a polygon, a spiral, a braid, a slotted tube, a filled tube, and combinations thereof.

In some embodiments of the invention, the medical device comprises a first swellable material and a second material, said first swellable material having different swelling characteristics than the second material. The second material may also be a swellable material or it may be a non-swellable material. The first swellable material and the second material may be located adjacent to each other or at separate locations within the medical device.

The first swellable material and the second material may have different rates of swelling and may therefore bend or otherwise change the shape of the medical device when contacted with water. This combination of swellable materials may be used to actuate that cutting and/or migration action of the medical device.

In an embodiment of the invention, the medical device comprises a bi-ring, said bi-ring comprising the first swellable material and a second material, the first swellable material expanding more by swelling than the second material, thereby creating a bending associated with the medical device. An example of this is shown in FIG. 9. Here the medical device (90) comprises a second swellable material (92) which has a lower swelling expansion than the inner first swellable material. When exposed to water, this combination of first and second swellable materials causes the medical device (92) to bend like a heated bi-metal construct. For a ring-structured medical device this means that the outer diameter of the ring will increase due to outwards-bending.

Multi-layer wires may be prepared using a stacking principle of the bi-ring medical device. An example is a bi-layer wire which comprises a layer of a first swellable material contacting a layer of a second material, the first swellable material expanding more by swelling than the second material, thereby creating a bending associated with the wire when the wire is exposed to water.

It may be preferred that the layer of the second material is a highly crystalline, non-swellable polymer having a high E-modulus. The layer of the first swellable material may e.g. contain one or more swelling-promoting agents.

When the layer of the first swellable material starts swelling, the volume change creates a systematic deformation of the wire or the medical device containing it. If the bi-layer wire is used in a braid-like or spiral-like structure and the layer of the second material is arranged so that it faces the vessel wall, swelling will make the medical device expand its diameter.

When a two-layer wire is prepared, the outer layer and the inner layer may be made using the same matrix polymer, thereby improving the compatibility of the two layers.

Layered wires may for example be obtained using a co-extrusion process or a solvent-based bonding process.

For example, two PLLA-based layers, one of them containing Bentonite, may be wetted by chloroform and afterwards pressed together. Due to the aggressive solvent property of chloroform both PLLA surfaces will immediately swell and bond together. Another possibility is to align the two layers in a first processing step and apply the solvent, e.g. chloroform, via the open capillary formed by the small gap between the contact surfaces of the two layers.

An alternative process is to prepare the first layer and to deposition the second layer on the first by solvent casting.

Multi-layer wires, such as 3-layer wires or 4-layer wires, are also envisioned as part of some embodiments of the present invention and these may be prepared using the same processes as the two-layer wires.

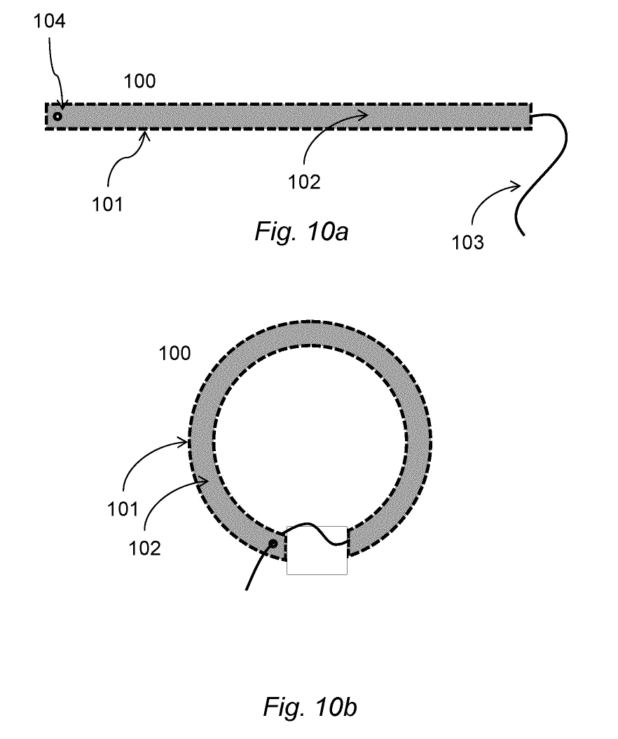

In some embodiments of the invention, the medical device comprises a filled tube, e.g. as illustrated in FIGS. 10a and 10b. Here the swellable material (102) of the medical device (100) is held by a tube-like membrane (101). The tube-like membrane (101) provides the medical device (100) with structure and the swellable material (102) provides the medical device (100) with expansion upon contact with water. The filled tube may comprise one of the locking mechanisms mentioned herein, e.g. a hole (104) and a wire (103) for ties the two ends of the filled tube (100) together. The swellable material (102) may e.g. comprise swellable powder, granulate and/or hydrogel. The swellable material (102) and the membrane material (101) may e.g. substantially or entirely consist of biodegradable materials.

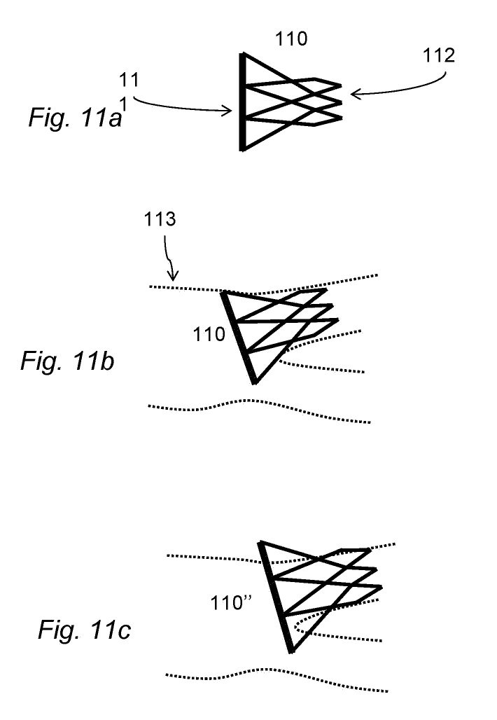

A further embodiment of the invention is depicted in FIG. 11. Here the medical device (110) comprises a biodegradable ring (111) containing swellable material which ring is attached to a non-biodegradable part (112). The non-biodegradable part is used for fixing the medical device at the target area (113). When implanted, the ring will expand and grow through the wall of the target area, e.g. the atrial wall. The non-biodegradable part (112) may preferably be sized to fit into the target area. After a while, the ring (111) will be biodegraded leaving the non-biodegradable part (112) in the target area (113).

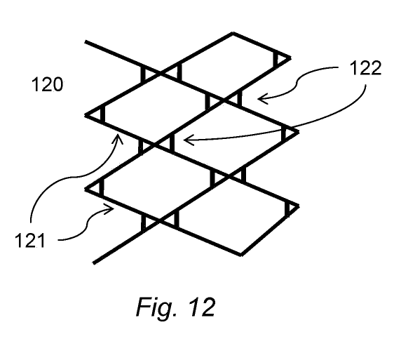

Another embodiment of the invention is shown in FIG. 12. The main body (121) of this medical device (120) has a braid-like structure and is made of a non-swellable material. The medical device furthermore comprises elements of swellable material (122) between the wires of the braid-like structure. When contacted with water the elements of swellable material will expand and make the braid-like medical device expand as well. The medical device of FIG. 12 may be operated like the medical device of FIGS. 8a and 8b.

The swelling kinetic can be customized using barriers or matrices partly or completely covering the swellable material and thus controlling the access of water to the swellable material. Thus, according to some embodiments of the invention, the medical device comprises a barrier or matrix which at least partly covers the medical device or the swellable material thereof, said barrier arranged for regulating the access of water to the swellable material.

In some embodiments of the present invention, it is preferred that the onset of swelling of the swellable material is delayed in the range of 1 hour-1 month after insertion, preferably in the range of 1 day to 3 weeks and even more preferably in the range of 5 days to 2 weeks. The onset of the swelling may be determined as the time of incubation in isotonic phosphate buffer at 20° C. that is required for the medical device to expand 1% in at least one dimension, e.g. in its diameter or in its length. The expansion should be determined relative to the pre-swelling state of the medical device, e.g. its dry state.

The barrier or matrix may be arranged and configured to obtain such delay in swelling. An advantage of delayed swelling is that the medical device will be covered with endothelial cells once the swelling starts and therefore less likely to disintegrate in the target area before the cutting/migration action has been performed.

The barrier or matrix may have another role, namely to provide the medical device with mechanical structure and mechanical stability and to keep the swellable material in place. The barrier may e.g. comprise woven material, preferably in a mesh size sufficiently small to keep the swelling material in place. The barrier or matrix may also comprise a perforated polymer layer. The perforations may e.g. be performed using a laser or pins. The barrier or matrix may also comprise a permeable polymer layer or a semi-permeable polymer layer. The polymer layer may e.g. be a thin tube, a membrane or a foil.

The barrier may comprise one or more coating layers. An increasing number of coating layers typically results in a decreasing water permeability of the barrier and thus a decreasing rate of swelling.

Useful coating layers may e.g. be applied using chemical or physical vapour deposition methods.

In an embodiment of the invention, the barrier is a semi-permeable barrier, i.e. permeable for water and small ions but impermeable for larger molecules. Semi-permeable barriers are capable of building up osmotic pressures inside medical devices according to some embodiments of the invention.

The barrier may comprise one or more biodegradable layers, which initially prevents or reduces access of water to the swellable material, but which degrades after a predetermined period of time in the body to allow the water to be absorbed.

The barrier may e.g. comprise a closed, tube-like membrane, which contains swellable material in its interior.

In some embodiments of the invention, the medical device is restrained in a temporary delivery shape. This may be particularly useful for ready-to-use medical devices.

### FURTHER MODES OF ACTUATION

In some embodiments of the invention, the medical device comprises at least one further actuation component. The further actuation component may e.g. comprises one or more actuation components selected from the group consisting of an expanding balloon, a thermal expansion component such as a shape memory material, a spring, a mechanical unfolding component, and any combination thereof.

The combination of an expanding balloon and a swellable material is shown in FIG. 7.

The swellable material and the further actuation component may be arranged to actuate the change of shape concurrently, i.e. the further actuation component changes shape.

The change of shape caused by swelling may be a relatively slow process and it may therefore be preferred that the swellable material and the further actuation component are arranged to actuate the change of shape sequentially. For example, the further actuation component may be adapted to actuate the change of shape from said temporary delivery shape to the expanded delivered shape, and the swellable material may be adapted to actuate the change of shape from said temporary delivery shape to said further expanded shape.

In some embodiments of the invention, the medical device comprises an endotheliasation agent. It is particularly preferred that the endotheliasation agent comprises a capturing ligand which specifically binds endothelial progenitor cells and/or endothelial cells. The capturing ligand is may e.g. comprise an antibody, a Fab fragment, a receptor, or a combination thereof. Useful capturing ligands are described in WO 00/168158, which is incorporated herein by reference in its entirety for all purposes.

WO 00/168158 discloses compositions and methods for producing a medical device such as a stent or a synthetic graft, coated with a matrix and an antibody which reacts with an endothelial cell antigen. The matrix coating the medical device may be composed of synthetic material, such as polyurethane, poly-L-lactic acid, cellulose ester or polyethylene glycol. The matrix may be composed of naturally occurring materials, such as collagen, fibrin, elastin, amorphous carbon. The matrix may be composed of fullerenes which range from about C60 to about C100. The antibodies promote adherence of endothedial cells on the medical device. The antibodies may be mixed with the matrix or covalently tethered through a linker molecule to the matrix. Following adherence to the medical device, the endothelial cells differentiate and proliferate on the medical device. The antibodies may be different types of monoclonal antibodies.

However, the use of such endotheliasation agents, especially as described in WO 00/168158, is not known to anchor a medical device in a tissue or vessel wall. Hence, in some embodiments of the present invention, comprising an endotheliasation agent, the medical device may be anchored in an advantageous way at the location of delivery thereof. In more detail, the anchoring of the medical device in the body is promoted by means of the endotheliasation agent. Once anchored in the tissue by a layer of endothelia, the medical device is separated from the blood flow, which is advantageous. A cutting and/or migrating effect is not hindered by the dynamics of surrounding blood, as the medical device is firmly anchored and cannot be flushed away or dislocated, which is an undesired effect.

In some embodiments of the invention, the medical device in addition comprises at least one drug. The at least one drug may be comprised in a coating or as layers within said medical device.

Examples of useful drugs are ciclosporin, taxiferol, rapamycin, tacrolimus, alcohol, glutaraldehyde, formaldehyde, and proteolytic enzymes like collagenase. Collagenase is effective in breaking down tissue and especially fibrin tissue, which is otherwise difficult to penetrate. Therefore, covering the surface of the medical device with collagenase would particularly speed up the process of penetrating tissue. The drugs may be attached to the surface of the medical device according to well-known methods of attaching drugs to medical devices. One such method is embedding drugs into or under layers of polymers, which cover the surface. Of course, other methods may be used. Similarly, drugs preventing thrombosis and increasing in-growth of endothelium on the endothelial surface after penetration of the medical device may be attached to the medical device. Such drugs could be e.g. Endothelium Growth Factor, and Heparin. Also, other drugs designed to treat arrhythmias may be attached to the medical device surface. Such drugs are e.g. amiodarone and sotalol.

The at least one drug may also comprise a fibrotic agent, such as collagen. The fibrotic agent may be arranged in such a manner that it is released with a delay during the cutting and/or migrating of the medical device. The delay may be controlled by means of a barrier or matrix that release of the fibrotic agent is provided upon complete endotheliasation of the medical device, such that a release of the fibrotic agent to the blood flow is prevented.

The medical device according to some embodiments of the invention may be structured and arranged to be inserted into a body vessel and to subsequently change shape, wherein the device is structured and arranged to change shape to extend at least partly outside the perimeter or orifice of an outer wall of said vessel in said further expanded shape.

In some embodiments of the invention, the medical device is inserted using a delivery system. A possible delivery system comprises an outer tube (pull tubing) wherein the compressed/or non-expanded medical device is positioned. Furthermore, the delivery system may have an inner tubing (push tubing).

The medical device is released from the system by holding the push tubing fixed and pulling in the pull tubing until the medical device is free of the system. Alternatively the medical device is released by holding the pull tubing fixed and pushing on the push tubing until the medical device is free of the system. For complex medical devices these actions can be done sequentially or combined. A further inner tube is available as a guide wire. Typically, the tube is made of plastic or braided stainless steel.

Yet an aspect of the present invention relates to a kit of shape-changing medical devices as described herein for treatment of disorders in the heart rhythm regulation system, said kit comprising: said shape-changing medical devices, which each has a first delivery and a second delivered shape, wherein the device in the first shape has such dimensions as to be insertable to a desired position within the vascular system, and wherein the device is capable of changing shape to substantially the second shape when located at said desired position, which strives to a diameter that is larger than the diameter of the vessel at the desired position, whereby the device will become embedded into the tissue surrounding the vessel at the desired position and create fibrosis and/or create scar tissue in said tissue in order to prevent it from transmitting electrical signals, wherein at least one of the shape-changing devices is adapted to be inserted to a desired position at the orifice of a pulmonary vein in the heart, the pulmonary vein adjacent said orifice, or inside the right or left atrium and at least one of the shape-changing medical devices is adapted to be inserted to a desired position in the coronary sinus, and wherein said medical devices comprise a swellable material which is capable of actuating said change of shape via swelling due to water absorption of said swellable material.

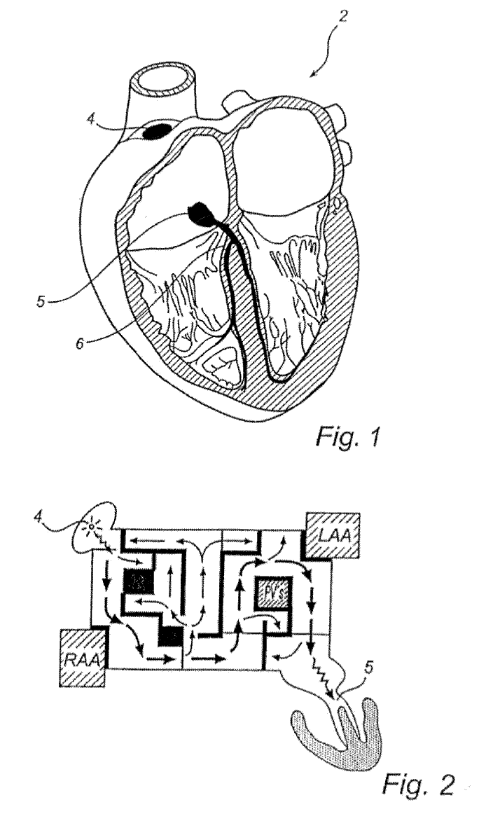

Referring to FIGS. 1-3, the problems of disorders relating to the heart rhythm regulation system and the leading current method of treating these problems will be described. In FIG. 1, a heart 2 is shown and the controlling of the heart rhythm is indicated. The heart rhythm is normally controlled from the sinoatrial node 4. The sinoatrial node 4 transmits electrical signals which are propagated through the heart wall by means of special cells forming an electrical pathway. The electrical signals following the electrical pathway will coordinate the heart muscle cells for almost simultaneous and coordinated contraction of the cells in a heart atrium and heart ventricle. The normal conduction of electrical impulses through the heart starts in the sinoatrial node 4, travels across the right atrium, the atrioventricular node 5, the bundles of His 6 and thereafter spread across the ventricular muscle mass. In a disordered situation, electrical signals are started in heart cells outside the sinoatrial node 4, in so called ectopic sites. These electrical signals will disturb the coordination of the heart muscle cells. If several ectopic sites are present, the signal transmission becomes chaotic. This will be the cause of arrhythmic diseases, such as atrial fibrillation and atrial flutter.

An existing method for treating these diseases is based on isolating the ectopic sites in order to prevent the electrical signals started in these ectopic sites to propagate in the heart wall. Thus, the heart wall is cut completely through for interrupting the coupling between cells that transmit erratic electrical signals. The thus created lesion will be healed with fibrous tissue, which is unable to transmit electrical signals. Thus, the path of the electrical signals is blocked by this lesion. However, since the location of the ectopic sites may not always be known and may be difficult to determine or since there might be multiple ectopic sites, a special cutting pattern has been developed, which will effectively isolate ectopic sites. Thus, the same pattern may always be used regardless of the specific locations of the ectopic sites in each individual case. The procedure is called the “Maze”-procedure in view of the complicated cutting pattern. In FIG. 2, the Maze-pattern is illustrated.

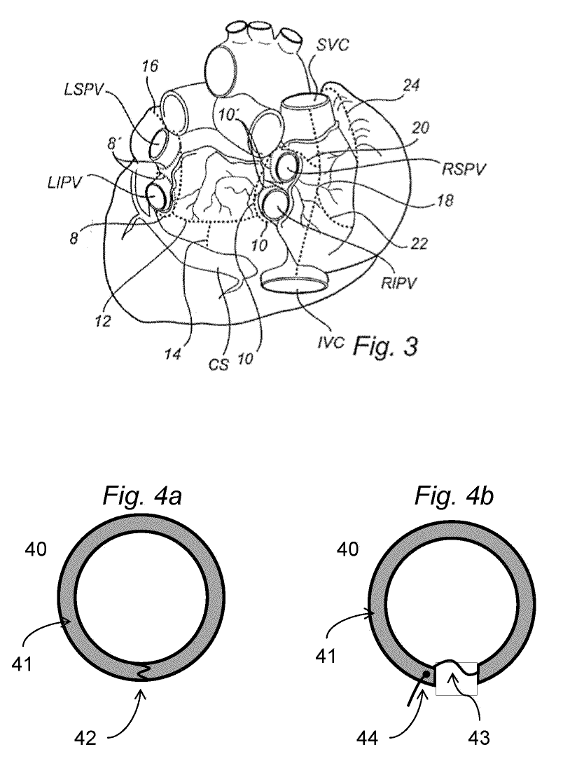

However, as is evident from FIG. 2, the cutting pattern is extensive and complex and requires a difficult surgery. Thus, the Maze-pattern has been evolved in order to minimize the required cuttings and simplify the pattern as much as possible. Currently, a Maze III-pattern is used, as shown in FIG. 3. This pattern is not as complicated, but would still effectively isolate the ectopic sites in most cases. The Maze III-pattern comprises a cut 8 around the left superior pulmonary vein (LSPV) and the left inferior pulmonary vein (LIPV) and a corresponding cut 10 around the right superior pulmonary vein (RSPV) and the right inferior pulmonary vein (RSPV); a cut 12 connecting the two cuts 8 and 10 around the pulmonary veins (PV); a cut 14 from this connecting cut to the coronary sinus (CS); a cut 16 from the left PVs to the left atrial appendage; a cut 18 from the inferior vena cava (IVC) to the superior vena cava (SVC); a cut 20 connecting the cut 10 around the right PVs and the cut 18 between the IVC and the SVC; a cut 22 from the cut 18 between the IVC and the SVC along the right lateral atrium wall; and a cut 24 isolating the right atrial appendage. Thus, a pattern, which is less complex and which effectively isolates the ectopic sites, has been established. In some cases, all cuts may not be needed. For example, the occurrence of ectopic sites often starts around the orifices of the PVs and, therefore, it may be sufficient to make the cuts 8, 10 around the PVs. Further, as indicated with the lines 8′ and 10′, the cuts around the PVs may be done along each PV orifice instead of in pairs.

A plurality of embodiments of the medical device described herein may intraluminally be placed at the positions to achieve a Maze-III like treatment without the need of open chest surgery.

A further aspect of the invention relates to a method for treatment of disorders in the heart rhythm regulation system using a medical device comprising a swellable material, the method comprising

inserting the medical device in a temporary delivery shape through the vascular system into a body vessel adjacent to the heart and/or into the heart;

allowing said swellable material to swell by absorbing water, said swelling at least contributing to changing the shape of the medical device, from said temporary delivery shape via an expanded delivered shape to a further expanded shape, extending at least beyond an outer surface of said tissue and/or vessel, thereby

creating cutting action configured for cutting said heart tissue and/or said body vessel, thereby

reducing undesired signal transmission in said heart tissue and/or said body vessel by cutting said heart tissue and/or said body vessel by means of the medical device configured therefore.

The step of inserting preferably comprises inserting said medical device into a desired position in the coronary sinus, in any of the pulmonary veins, in the superior vena cava, in the inferior vena cava, or in the left or right atrial appendage.

The method may furthermore comprise inserting at least another medical device into another of the plurality of desired positions. For example, the method may comprise inserting a medical device into each of the plurality of desired positions.

In some embodiments of the invention, the method furthermore comprises restraining the medical device in a temporary delivery shape during the insertion of the medical device.

The restraining may e.g. be accomplished using a medical device including a biodegradable barrier which restrains the medical device in its temporary delivery shape. Once introduced into the body, the biodegradable barrier dissolves, whereby the restraint is removed and the change of shape can take place.

Restraining may also comprise keeping the medical device inside a tube, which is removed or dissolved once the medical device is in place. This is for example useful when the medical device comprises a further actuation component such as a spring.

Another example of restraining comprises cooling the medical device. This is e.g. useful when handling medical devices including shape memory material.

The method involves restraining the medical device, it normally also comprises releasing the restrain on the medical device when it has been inserted into the desired position, thus allowing said change of the shape of the medical device.

The method may furthermore comprise eluting at least one drug from said medical device. The at least one drug is for instance a fibrotic agent.

The medical device may in some embodiments alternatively or in addition comprise an endotheliasation agent, thereby accelerating the overgrowth of the device with endothelial cell and anchoring the medical device in the wall of the body vessel and/or in the heart tissue.

It is preferred that the endotheliasation agent comprises a capturing ligand which specifically binds endothelial progenitor cells and/or endothelial cells.

A further aspect of the invention relates to a method of producing a medical device, the method comprising the steps of:
providing a swellable material
transforming said swellable material into a medical device configured for reducing undesired signal transmission in a body vessel adjacent to the heart and/or a heart tissue by isolating electrical propagation thereof by cutting and/or migrating into said vessel and/or tissue.

The swellable material may e.g. be provided as a wire.

In an embodiment of the invention, the step of transforming comprises winding the wire to form a medical device comprising a spiral-like structure.

In another embodiment of the invention, the step of transforming comprises braiding the wire to form a medical device comprising a braid-like structure.

The step of transforming may furthermore comprise a thermal treatment of the braid-like structure or the spiral-like structure to heat set said structure.

The method of preparing the medical device may furthermore comprise one or more steps selected from the group consisting of applying a coating layer or a barrier to the surface of the medical device, applying an endothelialisation agent to the surface of the medical device, and/or sterilising the medical device.

Ranges mentioned in this present specification may comprise the ranges in approximation or the ranges with exact boundary values.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present invention has been described above with reference to specific embodiments. However, other embodiments than the above described are equally possible within the scope of the invention. The different features and steps of the invention may be combined in other combinations than those described. The scope of the invention is only limited by the appended patent claims.

### EXAMPLES

### Example 1

### Swellable PLLA/PEG Hydrogel

A swellable PLLA/PEG hydrogel may be prepared as follows.

40 g of PEG 4000 (polyethylene glycol) with an approx. MW of 3500 to 4500 is dried at 40° under vacuum for one day. 3 g of 1.4 Diisocyanatobutane is added to the vacuum-dried PEG. The mixture is heated up to 70° C. and stirred, after melting above 55° C., for approx. 4 hours in an inert and dried atmosphere. Dried low molecular weight PLLA (poly L-lactide), for example Fluka 94829 Poly(L-lactide) is dissolved in dried CHCL3. To use a medical grade specified PLLA oligomer would be preferred due to possible impurities caused by the initiator system used at the polymer synthesis step. Ultrasonic treatment of the PLLA solution may be used to cut down molecular weight further. The preferred end point of a optional ultrasound treatment of the PLA solution may be determined by viscosity based measurements.

An (OH)-end group determination/titration should be used to identify a proper ratio of oligomers relative to the initial PEG content, preferably in a relation of 2 Mol PLLA oligomer to 1 MOL PEG.

The PLLA polymer or oligomer solution is used to dissolve the isocyanate end captured PEG. The solution may be diluted by additional dry CHCl3 to obtain a viscosity which is may be spray dried. The solution is stirred for 1 h at room temperature. Approx. 1 ml of H20 is added to the polymer solution in order to destroy free isocyanate functions. The solution is stirred until it looks homogenous.

The solution is then spray dried and subsequently vacuum dried at a temperature around 160° C. in order to reduce the content of residual butanediamine. The resulting hydrogel contains covalently cross-linked blocks of PLA oligomers (R1) and PEG (R2).

The hydrogel may e.g. have the structure R1-NO-(CH2)4-NO-R2-NO-(CH2)4-NO-R1. The degradation products of the hydrogel will all be based on hydrolyzed monomers like lactide acid, ethylene glycol and a minor content of butanediamine. All these products are suitable to be released in body environment.

The described hydrogel can be use as an additive to modify PLA to get desired swellable properties. The dry modified Pla may be extruded into a wire having an outer diameter of approx. 0.4 mm by conventional extrusion.

### Example 2

### Spiral Medical Device

A spiral medical device may be prepared from the hydrogel wire prepared in Example 1. Such a medical device has been schematically illustrated in FIGS. 6a and 6b.

The wire is wound around a tubular core having an outer diameter of 19 mm, fixed to the core at and the heat set via heat treatment at 60° C. for 4 h.

After the heat treatment the core is removed and the medical device is cut into a suitable length, typically approx 50 mm. The medical device can be compressed and loaded into a catheter and is self-expanding once is release at its target location.

Absorption of water by the swellable material of the wire is expected to cause the diameter of the spiral to expand at least 10 mm.

1. A medical device configured for reducing undesired signal transmission in a body vessel adjacent to the heart and/or a heart tissue by isolating electrical propagation thereof by cutting and/or migrating into said vessel and/or heart tissue, wherein said medical device is configured for cutting and/or migrating by changing its shape, and
wherein said medical device comprises a swellable material which is capable of actuating said change of shape via swelling due to water absorption by said swellable material.

2. The medical device according to claim 1, wherein the device is structured and arranged to be inserted in a temporary delivery shape through the vascular system into a body vessel adjacent to the heart and/or into the heart and to be subsequently subjected to a change of shape, from said temporary delivery shape via an expanded delivered shape to a further expanded shape, extending at least beyond an outer surface of said vessel and/or tissue, in order to create cutting action configured for cutting said heart tissue and/or said body vessel.

3. The medical device according to claim 1, wherein at least one dimension of the medical device has expanded in the range of 5-500% relative to its pre-swelling state after having been incubated in isotonic phosphate buffer at 20° C. for one week.

4. The medical device according to claim 1, wherein at least two dimensions of the medical device have expanded in the range of 5-500% relative to its pre-swelling state after having been incubated in isotonic phosphate buffer at 20° C. for one week.

5. The medical device according to claim 3, wherein the expansion in at least one dimension or at least two dimensions comprise radial expansion.

6. The medical device according to claim 1, wherein the swellable material comprises a swellable polymer.

7. The medical device according to claim 1, wherein the swellable polymer comprises a polar non-charged polymer.

8. The medical device according to claim 1, wherein the swellable polymer comprises a polyelectrolyte.

9. The medical device according to claim 1, wherein the swellable polymer comprises a swelling-promoting additive.

10. The medical device according to claim 9, wherein the swelling-promoting additive comprises an inorganic additive.

11. The medical device according to claim 9, wherein the swelling-promoting additive comprises an organic additive.

12. The medical device according to claim 1, wherein the swellable material furthermore comprises a polymer matrix mixed with one or more swelling promoting additives.

13. The medical device according to claim 1, wherein the medical device comprises a ring-like structure.

14. The medical device according to claim 1,
wherein the medical device comprises a polygon-like structure.

15. The medical device according to claim 1, wherein the medical device comprises a spiral-like structure.

16. The medical device according to claim 1, wherein the medical device comprises a slotted tube.

17. The medical device according to claim 1, wherein the medical device comprises a filled tube.

18. The medical device according to claim 1, wherein the medical device comprises a first swellable material and a second material, said first swellable material having different swelling characteristics than the second material.

19. The medical device according to claim 1, wherein the medical device comprises a bi-ring, said bi-ring comprising the first swellable material and a second material, the first swellable material expanding more than the second material, thereby creating a bending associated with the medical device.

20. The medical device according to claim 1, wherein the medical device comprises a barrier at least partly covering the medical device or the swellable material thereof, said barrier arranged for regulating the access of water to the swellable material.

21. The medical device according to claim 20, wherein the barrier comprises a tube-like membrane containing swellable material.

22. The medical device according to claim 1, wherein the medical device is restrained in a temporary delivery shape.

23. The medical device according to claim 1, wherein the medical device comprises a further actuation component.

24-53. (canceled)
